(19) United States
(12) Patent Application Publication
(10) Pub. No.: US 2022/0280525 A1
(43) Pub. Date: Sep. 8, 2022

(54) RAC INHIBITION AS A NOVEL THERAPEUTIC STRATEGY FOR EGFR/HER2 TARGETED THERAPY RESISTANT BREAST CANCER

(71) Applicant: UNIVERSITY OF PUERTO RICO, San Juan, PR (US)

(72) Inventors: Luis D. BORRERO-GARCÍA, San Juan, PR (US); Suranganie DHARMAWARDHANE FLANAGAN, San Juan, PR (US)

(21) Appl. No.: 17/685,174

(22) Filed: Mar. 2, 2022

Related U.S. Application Data
(60) Provisional application No. 63/156,160, filed on Mar. 3, 2021.

(51) Int. Cl. A61K 31/5377; A61K 31/4192; A61K 31/517; A61K 45/06; A61P 35/00

(52) U.S. Cl. CPC A61K 31/5377; A61K 31/4192; A61K 31/517; A61K 45/06; A61P 35/00

(57) ABSTRACT

Targeted therapies are available for cancers expressing oncogenic epidermal growth receptor (EGFR) and (or) human EGFR2 (HER2), however acquired or intrinsic resistance often confounds therapy success. Common mechanisms of therapy resistance involve activating receptor point mutations and (or) upregulation of signaling downstream of EGFR/HER2 to Akt and (or) mitogen activated protein kinase (MAPK) pathways. However, additional pathways of resistance may exist thus, confounding successful therapy. To determine novel mechanisms of EGFR/HER2 therapy resistance in breast cancer, Gefitinib® or Lapatinib® resistant variants were created from SKBR3 breast cancer cells. Syngenic therapy sensitive and resistant SKBR3 variants were characterized for mechanisms of resistance by mammosphere assays, viability assays, and western blotting for total and phospho proteins. Combinations of treatments focused on RGFR/HER2 and Rac inhibitors (1,5-disubstituted 1, 2, 3-triazoles, e.g. Ehop-16 and MBQ-167) are proposed as viable strategies for treatment of EGFR/HER2 targeted therapy resistant breast cancer.

### CROSS REFERENCE TO RELATED APPLICATION

This application claims the benefit of priority under 35 U.S.C. § 119(e) to U.S. Provisional Patent Application No. 63/156,160, filed Mar. 3, 2021. The disclosure set forth in the referenced application is incorporated herein by reference in its entirey.

### GOVERNMENT SUPPORT

This invention was made with government support under grant nos. NIH/NIGMS SC3GM094824 and NIH/NIGMS P20 GM103475, awarded by the National Institutes of Health, and grant no. W81XWH2O10041, awarded by US Army Breast Cancer Research Program. The government has certain rights in the invention.

### BACKGROUND

Aggressive breast cancers overexpress Epidermal Growth Factor Receptor (EGFR) family members. ˜25% of breast cancer patients overexpress human epidermal growth factor receptor 2 (HER2) and ˜15% overexpress the EGFR1 isoform. EGFR/HER2 overexpression in breast cancer increases breast cancer malignancy by upregulated cancer cell survival, invasion and metastasis, maintenance of stem cell-like tumor cells, and resistance to targeted therapies. Therefore, a number of EGFR- and HER2-targeted therapeutics have been developed. These include small molecules that inhibit the tyrosine kinase domain of the EGFR such as gefitinib (EGFR1) and lapatinib (EGFR1 and HER2). However, the effectiveness of EGFR tyrosine kinase inhibitors (TKI)s in the clinic has been greatly impaired by the development of de novo or acquired resistance. Specifically, trials with gefitinib in breast cancer resulted in poor clinical response indicating that intrinsic resistance to gefitinib, and therefore, to TKIs, is common in breast cancer. Similarly, the initial success of lapatinib, which was developed as an ATP-competitive reversible EGFR/HER2 inhibitor, has also been marred by intrinsic and acquired therapy resistance. Consequently, it is crucial to elucidate the mechanisms of EGFR/HER2 therapy resistance, and to develop targeted strategies to reverse such resistance.

Several mechanisms of acquired resistance to TKIs have been reported, including EGFR gene mutations, activation of the phosphoinositide 3-kinase (PI3K)/Akt/mammalian target of rapamycin (mTOR) pathway and the Ras/MAPK pathway (Liu et al., 2018), as well as epithelial to mesenchymal transition (EMT), where acquisition of cancer stem cell-like phenotypes is associated with resistance to TKIs (de Melo et al., 2016).

Metastasis, that is, when the cancer cells undergo EMT and migrate to establish secondary tumors at distant vital sites, remains the major cause of death from breast cancer. Recent studies have shown that therapy resistant breast cancer cells possess more mesenchymal and stem cell-like properties and invade the circulatory system using migratory and invasive properties. After they are in the circulatory system, the therapy resistant cells can circulate in the blood or lie dormant in the bone marrow and distant organs, while retaining the capacity for self-renewal. Therefore, understanding the mechanisms of resistance leading to the acquisition of EMT and migratory and stem cell-like properties is highly relevant for effective breast cancer cure.

The EGFR (ErbB) family members are central transducers of a myriad of cellular signaling cascades that drive cancer progression. Specifically, the EGFR type II (HER2) may heterodimerize with the other three members of the family (EGFR1, EGFR3 and EGFR4) coordinating a series of pathways that lead to cell survival, proliferation, and invasion/migration.

The overexpression of EGFR family members has been observed in more than 20% of invasive breast carcinomas, and this amplification is associated with increased metastatic potential. Therefore, anti-EGFR therapy is considered a viable targeted strategy for cancers that overexpress these receptors. The use of lapatinib, a dual EGFR/HER2 therapeutic, has improved breast cancer patient survival when used in combination with HER2-targeted therapeutics such as trastuzumab. However, the failure in the approval of gefitinib, and the resistance by many patients to trastuzumab and lapatinib, remains a challenge in using these therapeutics. Therefore, the identification of resistance pathways and the development of new approaches to enhance patient response to TKIs is a critical objective, where combination therapy targeting the downstream signaling pathways is a viable strategy.

### SUMMARY

The combination of Rac inhibitors EHop-016 and MBQ-167 and EGFR/HER2 targeted therapy in breast cancer cells was tested, and found to inhibit viability and induce apoptosis of otherwise therapy resistant cells. “Therapy” is defined as, for example, gefitinib and lapatinib treatments. These therapies reduce mammosphere formation in SKBR3 sensitive breast cancer cells, but not in the therapy resistant variants. These results indicate that therapy resistant cells have enhanced mesenchymal and cancer stem cell-like characteristics. The therapy resistant variants did not show significant changes in known therapy resistant pathways of AKT and MAPK activities that are downstream of EGFR/HER2. However, these resistant cells exhibited elevated expression and activation of the small GTPase Rac, which is a pivotal intermediate of GFR signaling in EMT and metastasis.

Therefore, Rac inhibition is proposed as a viable strategy for treatment of EGFR/HER2 targeted therapy resistant breast cancer.

To elucidate novel mechanisms and therapeutic strategies to overcome EGFR/HER2 therapy resistance, syngenic SKBR3 human breast cancer cell variants resistant to gefitinib (anti-EGFR) or lapatinib (anti-EGFR/HER2) were created. Therapy resistant variants exhibit a more aggressive mesenchymal phenotype with elevated viability/apoptosis and stem cell like activity, associated with increased expression and activity of the Rho GTPase Rac. Rac is a critical molecular switch activated by EGFR/HER2 signaling to regulate cell proliferation, survival, and migration, and thus EMT and metastasis. Consequently, Rac plays a significant role in resistance to EGFR/HER+breast cancer by acting downstream of EGFR/HER2 therapy resistance mechanisms such as Ras/MAPK and PI3-K/Akt signaling (Zhao et al., 2011). Herein, the potential for Rac inhibitors as targeted therapeutics for EGFR/HER2 therapy resistant breast cancer is demonstrated.

In conclusion, malignant cancer cells hijack alternate pathways to survive anti-EGFR/HER2 therapy and grow and migrate or stay dormant. The data presented here supports that Rac plays an integral role in the activation of EGFR/HER2 signaling during therapy resistance and that this increase in active Rac levels may promote cancer stem cell maintenance, as well as cell growth and survival. Therefore, novel therapies targeting Rac, such as EHop-016 and MBQ-167 (PCT/US2018/057148 and PCT/US2017/029921), are suggested as therapeutics to use individually or in combination with EGFR/HER2 therapy to treat resistant breast cancer.

### BRIEF DESCRIPTION OF THE DRAWINGS

FIG. 1A-1B. Viability of therapy sensitive and resistant variants in the presence of TKIs. (A) SKBR3 therapy sensitive cells, and variant resistant to 0.1 μM lapatinib, (B) SKBR3 therapy sensitive and variants resistant to 0.1 gefitinib or 0.5 μM gefitinib, were subjected to a MTT cell viability assay to determine the IC₅₀ by exposing the cells to different concentrations of TKIs gefitinib and lapatinib. % Cell viability in response to gefitinib or lapatinib is shown for the therapy sensitive and resistant variants. N=4±SEM

FIG. 2A-2G. EGFR and HER2 expression and phosphorylation in therapy sensitive and resistant variants. SKBR3 therapy sensitive or resistant (Gef.R 0.1 μM, Gef. R 0.5 μM and Lap. R 0.1 μM) cells treated with gefitinib or lapatinib for 24 h were lysed and western blotted for total and active (phospho) EGFR and HER2. (A) Representative western blots for pEGFR/EGFR (left) and pHER2/HER2 (right), with actin as a loading control, for cells treated with gefitinib or lapatinib for 24 h. (B) Fold change in EGFR and HER2 expression and phosphorylation for the therapy sensitive SKBR3 cells from positive bands quantified using image J software. (C) Representative western blots for pEGFR/EGFR and pHER2/HER2 in therapy sensitive (SKBR3) or resistant (Gef R, LapR variants, maintained in the indicated concentrations of gefitinib or lapatinib. (D) Fold change in EGFR expression, (E) Fold change in HER2 expression, (F) Fold change in EGFR phosphorylation, (G) Fold change in HER2 phosphorylation, N=3±SEM. ****=p≤0.001, ***=p≤0.005, **=p≤0.01, *=p≤0.05

FIG. 3A-3B. Apoptosis in therapy sensitive and resistant variants Apoptosis in therapy sensitive and resistant SKBR3 cell variants was detected by a Caspase-Glo 3/7 Assay. (A) Fold change in Caspase 3/7 activity in the therapy sensitive SKBR3 cell line following Gef or Lap treatment for 48 h compared to the vehicle controls. (B) Fold change in caspase 3/7 activity in the therapy resistant cell lines following treatment compared to non-treated cells. N=3±SEM, *=p≤0.05, ***=p≤0.005

FIG. 4A-4F. Stem cell-like characteristics in therapy resistant variants. Mammosphere formation efficiency (MFE) of SKBR3 therapy sensitive and resistant variants was calculated by dividing the number of mammospheres formed by the number of cells seeded per well and multiplied by 100 for percentage. (A) Representative micrographs of mammosphere forming units. Fold changes of percentage are shown in: (B) MFE in gefitinib and lapatinib treated therapy sensitive cells relative to vehicle treated cells. (C, D) MFE in therapy resistant cells treated with (C) gefitinib or (D) lapatinib, relative to vehicle controls. (E) MFE of therapy resistant variants relative to therapy sensitive cells with no treatment. (F) Representative western blots of cancer stem cell markers integrin (33, CD133, and Nanog in SKBR3 therapy sensitive and resistant variants. N=3±SEM, *=p≤0.05 and, =p≤0.001

FIG. 5A-5D. Akt and MAPK activities in therapy resistant variants. SKBR3 gefitinib and lapatinib sensitive and resistant cells were lysed and subjected to (A) western blotting for expression and activity of a Akt/p-Akt^{S473, T308}, (B) p44/42 MAPK/p-MAPK^{T202, Y204} using total or phospho-specific antibodies to the active sites. (C, D) Average integrated density of p-Akt/Akt (C) or p-P44/42 MAPK/P44/42 MAPK (D), as quantified from Image J analysis of positive bands from western blots. N=3

FIG. 6A-6F. Inhibition of upregulated Rac in therapy resistant variants. (A) Rac activation was determined by a pulldown assay using the p21-binding domain of p21-activated kinase (PAK) from lysates of therapy sensitive or resistant SKBR3 cells. Representative western blots for active Rac.GTP, total Rac, and actin are shown. (B) SKBR3 gefitinb and lapatinib sensitive and resistant cells were subjected to a MTT assay for cell viability following 24 h in the Rac inhibitor EHop-016 at 0, 5, or 10 μM. (C) SKBR3 lapatinib resistant cells were subjected to a MTT assay for cell viability following 24 h in vehicle (0), 0.1 μM lapatinib, 250 nM MBQ-167, or a combination of 0.1 μM lapatinib and 250 nM MBQ-167. (D) SKBR3 lapatinib resistant cells were subjected to a caspase 3/7 assay for apoptosis following 24 h in vehicle (0), 0.1 μM lapatinib, 250 nM MBQ-167, or a combination of 0.1 μM lapatinib and 250 nM MBQ-167. (E) MDA-MB-435 laptinib resistant HER2+ cells were treated with 0.1 μM lapatinib, 250 nM MBQ-167, or a combination of 0.1 μM lapatinib and 250 nM MBQ-167 for 48 h and cell viability quantified by a MTT assay; fold change in cell viability relative to vehicle is shown. (F) MDA-MB-435 trastuzumab resistant HER2+ cells were treated with 5 or 10 μg/ml trastuzumab, 250 nM MBQ-167, or a combination of 5 μg/ml trastuzumab and 250 nM MBQ-167 for 48 h and cell viability quantified by a MTT assay; fold change in cell viability relative to vehicle is shown. N=3±SEM *=p≤0.05, **=p≤0.01****=p≤0.001.

FIG. 7A-7D. Effect of MBQ-167 in combination with Trastuzumab on viability of Trastuzumab resistant SKBR3 cells. MDA-MB-435 Trastuzumab resistant HER2+ cells were treated with various concentrations of Trastuzumabb or MBQ-167 individually and in combination for 96h. Cell viability was quantified by a MTT assay; % cell viability relative to vehicle (100%) is shown; (A) 48 hrs; (B) 72 hrs; (C) 96 hrs; (D) 120 hrs.

FIG. 8A-8B. Effect of MBQ-167 in combination with Trastuzumab on apoptosis of Trastuzumab resistant SKBR3 cells. SKBR3 Trastuzumab resistant HER2+ cells were treated with various concentrations of Trastuzumabb or MBQ-167 individually and in combination for 96h. Cell viability quantified by a MTT assay; % cell viability relative to vehicle (100%) is shown: (A) 48 hrs; (B) 72 hrs.

### DETAILED DESCRIPTION

Development of Therapy Resistant Cell Variants

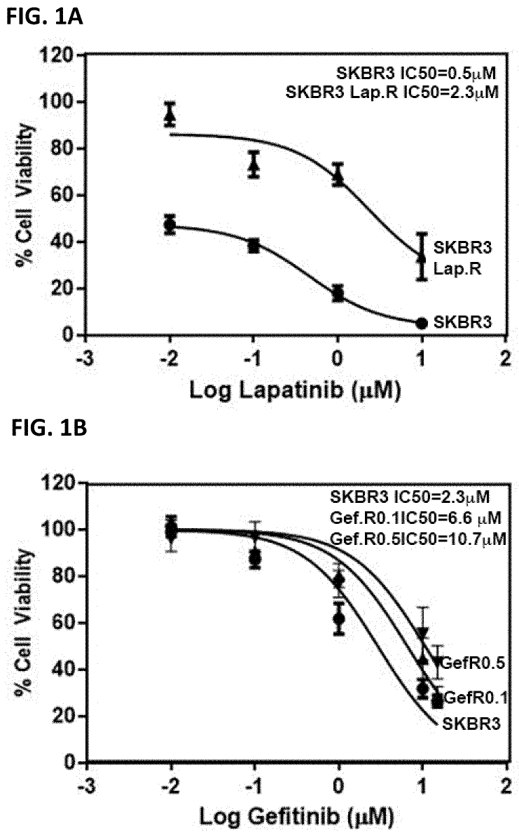

SKBR3 therapy sensitive EGFR and HER2 positive human breast cancer cells were created following exposure of the cells to gefitinib (0.1 or 0.5 μM) or lapatinib (0.1 μM). After six months of selection, the fold resistance was quantified as described in McDermott et al. (2014), using cell viability as a measure of resistance. Previous studies have established that a range of 2 to 5-fold resistance is required for a therapy resistant cell line to be considered clinically relevant. Cells that reach a fold resistance higher than 5-fold are designated as high laboratory-level resistant, and are useful for studies on mechanisms of resistance. The IC₅₀s for viability of the therapy resistant cell lines were divided by the IC₅₀ of the therapy sensitive cell line to obtain the fold resistance (FIG. 1A-1B). SKBR3 gefitinib resistant (Gef.R) cells at 0.1 μM, and lapatinib resistant (Lap.R) cells at 0.1 μM, demonstrated a fold resistance of 2.3 and 4.6 respectively, whereas Gef.R cells resistant to 0.5 μM gefitinib gave a fold resistance of 3.7. Therefore, the therapy resistant cell lines demonstrated clinically relevant fold resistance and were eligible for further investigation of the mechanisms of resistance.

EGFR/HER2 Activities in Therapy Resistant Breast Cancer Cells

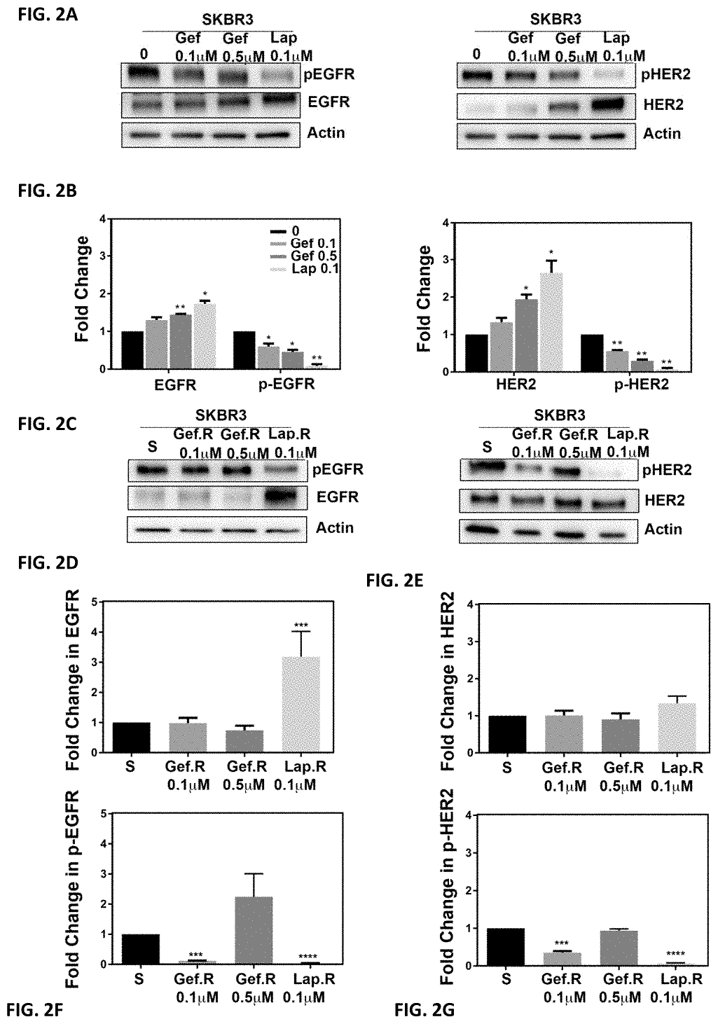

To determine the effectiveness of anti-EGFR therapy in the therapy sensitive and resistant variants, the levels of EGFR and HER2 and their activation (phospho (p)-EGFR and p-HER2) were evaluated in the therapy sensitive and resistant cells exposed to the same concentrations of gefitinb and lapatinib used to create the therapy resistant variants. Gefitinib reduced the phosphorylation of EGFR in sensitive SKBR3 cells at 0.1 μM and 0.5 μM concentrations (FIGS. 2A, B). Although gefitinib was developed to interact only with the ATP domain of EGFR, the results show that gefitinib also significantly decreased HER2 phosphorylation by 50-70% in a concentration dependent manner. Notably, the expression of total EGFR and HER2 was significantly elevated following 24 h in 0.5 μM gefitinib and 0.1 μM lapatinib treatments even in the sensitive SKBR3 cells, suggesting a possible mechanism of compensation (FIG. 2B).

The cell variants resistant to geftinib 0.1 μM and lapatinib 0.1 μM continued to respond to the drugs by decreased pEGFR and pHER2 levels demonstrating that the TKIs continued to act by inhibition of receptor phosphorylation (FIG. 2C). Of note are the SKBR3 Lap.R cells, which demonstrated increased EGFR expression compared to the sensitive cells, also suggesting a mechanism to compensate the decrease in activation (FIG. 2D).

However, Gef.R cells demonstrated no changes in expression of EGFR or HER2 (FIG. 2C, 2E). The cells resistant to 0.5 μM gefitinib demonstrated sustained phosphorylation of EGFR, suggesting a different mechanism of resistance than in the cells exposed to lower concentrations of gefitinib (FIG. 2F). Although gefitinib and lapatinib continued to inhibit EGFR and HER2 phosphorylation, and thus activation, these therapeutics did not affect the viability of the Gef.R and Lap.R cells, suggesting alternate mechanisms (FIG. 1).

Effect of EGFR Therapy on Apoptosis in Therapy Resistant Breast Cancer Cells

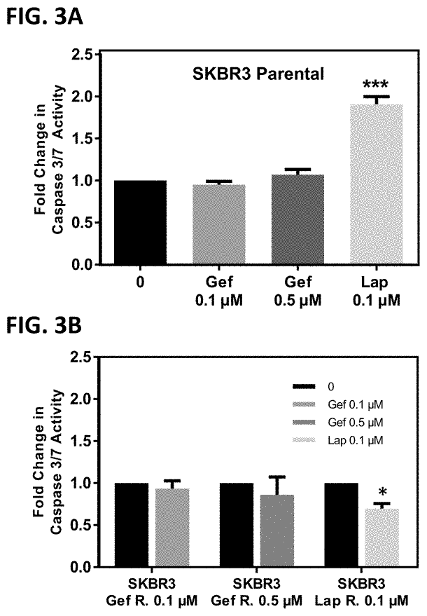

Previous studies have shown that lapatinib induces apoptosis in breast cancer cells. In order to test the hypothesis that lapatinib no longer induces apoptosis in the therapy resistant cell lines, a Caspase-Glo 3/7 assay was performed. The sensitive SKBR3 cells did not respond to gefitinib by apoptosis, but exhibited a 2-fold higher statistically significant increase in caspase 3/7 activity in response to 0.1 μM lapatinib, when compared to vehicle control (FIG. 3A). However, the lapatinib resistant variant showed a significant decrease in caspase 3/7 activity in response to lapatinib (FIG. 3B), suggesting that these cells are not only resistant to the treatment, but in the presence of the treatment, resistant cells may create an optimal environment for evading apoptosis.

Mammosphere Forming Efficiency of Therapy Resistant Breast Cancer Cells

Because cancer stem cells (CSCs) are an integral part of tumor progression, certain therapeutics can enrich the CSC population during acquisition of therapy resistance. Moreover, researchers have found that these CSCs share properties with metastatic cancer cells essential for providing a tumor microenvironment to support the growth of metastatic cells, along with evasion of cell death and increased survival. Additionally, the CSC hypothesis sustains that since normal stem cells tend to be quiescent, dormant CSCs may be resistant to therapies that target dividing cells.

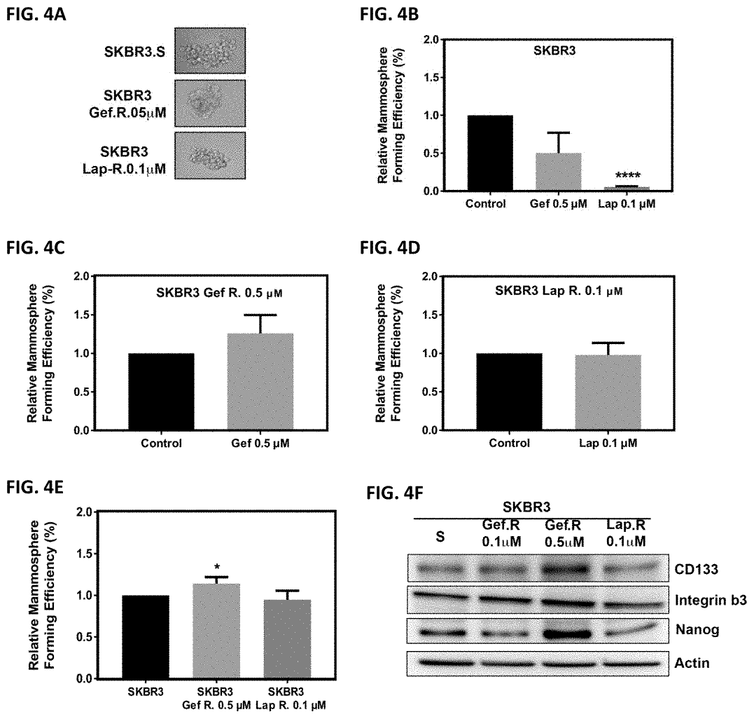

Therefore, to determine if the therapy resistant cells include a higher percentage of stem cell-like cells, a mammosphere assay was performed. Therapy sensitive SKBR3 cells showed a significant reduction in mammosphere formation after treatment with 0.5 μM gefitinib or 0.1 μM lapatinib (FIG. 4B). However, treatment with gefitinib or lapatinib had no significant effect on mammosphere formation in the therapy resistant variants (FIGS. 4C, 4D). Moreover, SKBR3 Gef.R cells resistant to 0.5 μM gefitinib showed a significant increase in mammosphere formation, and a correlative increase in the expression of stem cell markers such as integrin (33, CD133, and Nanog (FIGS. 4E, 4F). This result suggests that higher concentrations of gefitinib may be inducing different mechanisms of resistance and may provide a better environment for the survival and promotion of a stem cell-like phenotype in therapy resistant cells.

Molecular Mechanisms of EGFR Therapy Resistance in Breast Cancer Cells

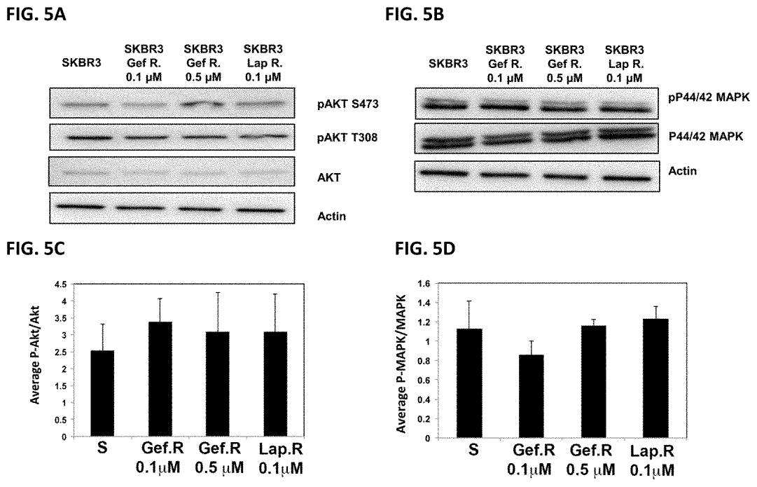

EGFR/HER2 therapy resistance is often due to upregulation of downstream signaling via phosphoinositide 3-kinase (PI3-K)/Akt, Ras/mitogen activated protein kinase (MAPK) or Rac/Cdc42/p21-activated kinase (PAK) pathways (Zhao et al., 2011). Therefore, levels of expression and activation of AKT and MAPK in the therapy resistant cells were compared to the therapy sensitive SKBR3 cell line, using antibodies to total and phospho (active) proteins. However, no significant changes were observed in the expression or activation of Akt or p42/44 MAPK in the therapy resistant variants compared to the therapy sensitive cell line (FIG. 5).

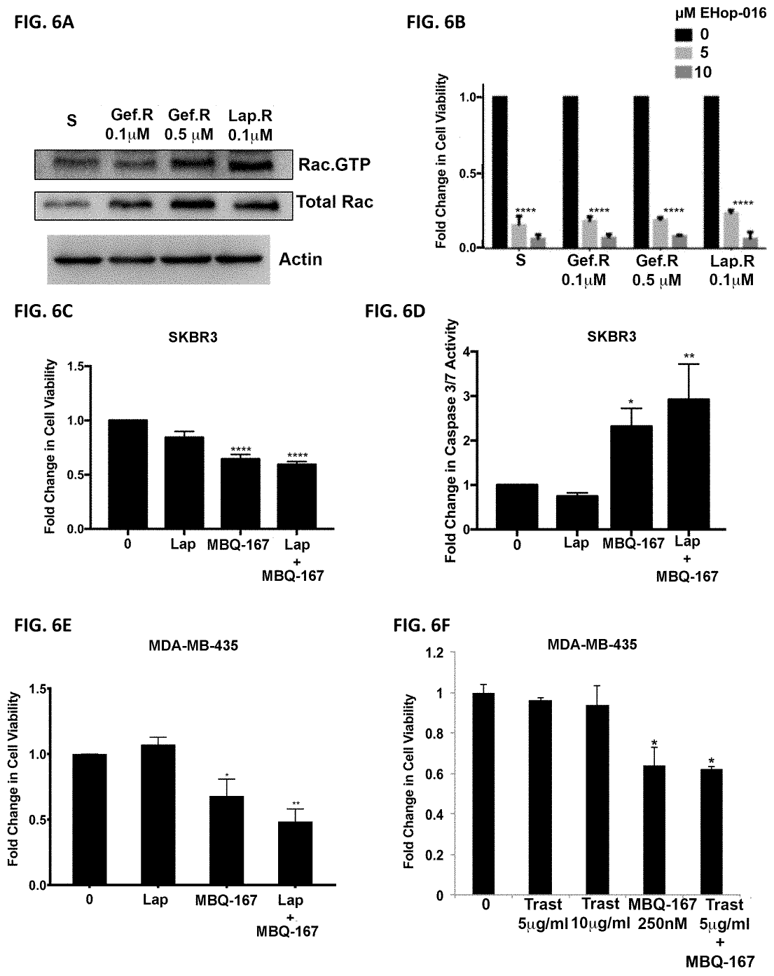

Because the Rho GTPase Rac signaling downstream of EGFR and HER2 has shown to contribute to EGFR/HER2 therapy resistance, expression and activation assay were performed to determine the role of Rho GTPases in the therapy resistant variants. Notably, compared to the therapy sensitive SKBR3 cell line, the therapy resistant cells demonstrated increased Rac expression, and thus, enhanced Rac activity (FIG. 6A). Moreover, no significant changes in expression were observed for the related Rho GTPases Rho and Cdc42 (Data not shown).

To determine whether the increased Rac activation contributed to therapy resistance, the effect of the Rac inhibitor EHop-016 (Humphries-Bickley et al., 2017) in therapy sensitive and resistant SKBR3 cells was tested. Results show a statistically significant decrease in cell viability at 5 and 10 μM EHop-016 for both sensitive and resistant cell variants.

An additional Rac inhibitor MBQ-167 was tested that was recently developed and characterized by the inventors as a more potent Rac and Cdc42 inhibitor compared to EHop-016 (Humphries-Bickley et al., 2017) in lapatinib resistant SKBR3 cells. Results show that while lapatinib did not affect the viability of the resistant variant, 0.5 μM MBQ-167 alone or in combination with 0.5 μM lapatinib significantly decreased cell viability by ˜40% (FIG. 6C). This reduction in cell viability resulted in apoptosis as seen by >2-fold increase in caspase 3/7 activity following MBQ-167 (0.25 μM) and an even higher significant increase in caspase activity when MBQ-167 (0.25 μM) was administered in combination with lapatinib (0.5 μM) (FIG. 6D). The gefitinib resistant SKBR3 variants also responded to the Rac inhibitor MBQ-167 by a similar phenotype of cell rounding, detachment from the substrate, and subsequent death, as previously reported in (Humphries-Bickley et al., 2017).

To determine if the effects of Rac inhibitors is a universal mechanism of resistance, the effect of Rac inhibition in a highly metastatic and therapy resistant variant of the MDA-MB-435 cell line, which was previously shown to demonstrate upregulated Rac compared to its less metastatic variants, was examined. As shown in FIGS. 6E, 6F, the metastatic MDA-MB-435 variant is insensitive to lapatinib and trastuzumab, a monoclonal antibody to the HER2 receptor, which is overexpressed in this cell line. However, the Rac/Cdc42 inhibitor MBQ-167 decreased the viability of this cell line by ˜40%. Combined lapatinib and MBQ-167 decreased cell viability further by ˜50%. MBQ-167 also inhibits MDA-MB-435 cell viability in the presence of trastuzumab, thus demonstrating its potential to inhibit therapy resistant cell viability. Thus, this data implicates Rac activation in EGFR/HER2 therapy resistance, and the potential of direct Rac inhibition by small molecule inhibitors to overcome TKI therapy resistance.

Demonstration of MBQ-167 efficacy in Lapatinib and Trastuzumab resistant MDA-MB-435 metastatic cancer cell line.

In Lapatinib resistant SKBR3 cells MBQ-167 (250 nM) reduced viability by 30% and apoptosis induction by 55%.

Similarly, in the MDA-MB-435 HER2++cell line, which is intrinsically resistant to Lapatinib and Trastuzumab, MBQ-167 significantly reduced viability in combination with Lapatinib.

Analysis of the effect of MBQ-167 to overcome Trastuzumab resistance in HER2++breast cancer cells.

SKBR3 HER2 positive trastuzumab sensitive cells were used to create a Trastuzumab (humanized monoclonal antibody to HER2) resistant syngeneic variant, where unlike the parental sensitive SKBR3 cell line, the Trastuzumab resistant cell variant was insensitive to increased Trastuzumab concentrations.

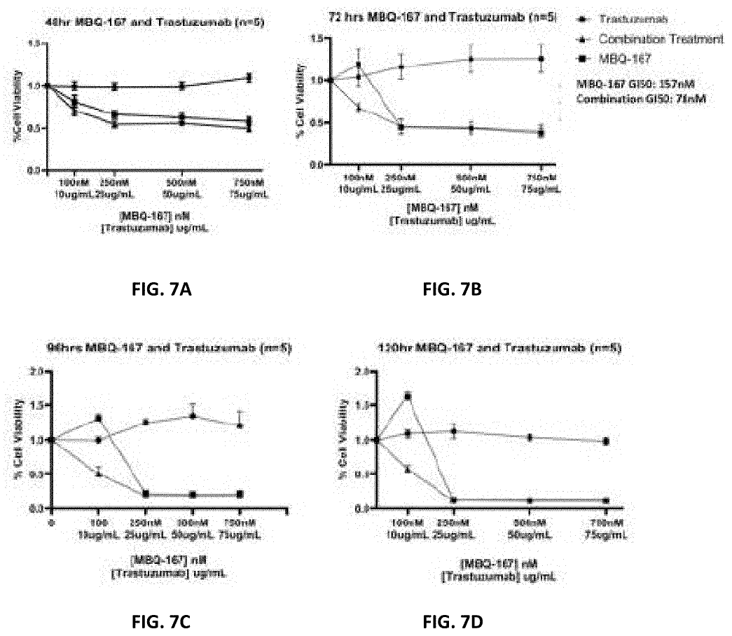

The efficacy of the Rac/Cdc42 inhibitor MBQ-167 to overcome Trastuzumab therapy resistance was tested in the Trastuzumab resistant SKBR3 breast cancer cell variants. Trastuzumab alone did not significantly affect cell viability in the resistance cell variant, while MBQ-167 decreased cell viability in a concentration and time-dependent manner, similar to the combination of MBQ-167 and Trastuzumab at various concentrations stating from their respective IC₅₀s in this cell line. i.e. 100 nM for MBQ-167 and 10 μg/ml for Trastuzumab. At 72 h, MBQ-167 resulted in a 50% decrease in viability, which was saturated at 250 nM. This effect was more dramatic at 96 and 120h, when viability was reduced by 90-100% at 250 nM MBQ-167 (FIG. 7).

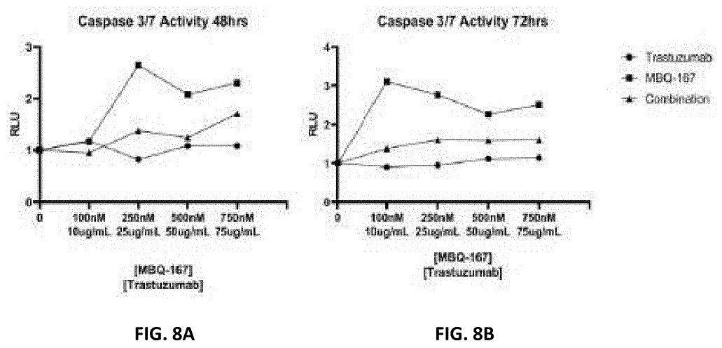

When apoptosis was analyzed by Caspase 3/7 assays, in the Trastuzumab resistant SKBR3 variant, MBQ-167 alone induced apoptosis in this cell line at concentrations ranging from 250-750 nM, while Trastuzumab alone had no effect. Intriguingly, MBQ-167 only partially overcame this resistance to apoptosis in the presence of Trastuzumab, indicating alternative resistance mechanisms in the Trastuzumab resistance cells (FIG. 8).

Conclusions are that in Trastuzumab resistant cells, MBQ-167 does not overcome therapy resistance to Trastuzumab; however, MBQ-167 is an excellent therapeutic alternative for use as single therapy to reduce viability and induce apoptosis in Trastuzumab resistant aggressive breast cancer.

Clinically relevant therapy resistant syngenic variants were successfully created from the SKBR3 therapy sensitive breast cancer cell line, and used as a model to investigate the mechanisms of resistance to both gefitinib and lapatinib. As observed, anti-EGFR therapy continues to inhibit EGFR and HER2 phosphorylation in the therapy resistant cells similar to the therapy sensitive cells. Interestingly, resistant cells that were exposed to the higher concentration (0.5 μM) of gefitinib did not respond via direct inhibition of EGFR or HER2 phosphorylation. This may be due to the acquisition of a resistant mutation, such as the EGFR T790M secondary mutation, which results in insensitivity to EGFR targeted therapy. In addition, the expression levels of EGFR and HER2 were higher in the therapy sensitive cells following TKI treatments, as well as in the lapatinib resistant cells (for EGFR), indicating that these cells may be synthesizing more receptors to compensate for the inactivation of this pathway. Also, even though it has been shown that gefitinib is a specific inhibitor of the tyrosine kinase domain of EGFR, data presented herein shows that gefitinib also decreases the phosphorylation of HER2. These effects on HER2 activity may be related to the heterodimerization complexes that occur between receptors (e.g. EGFR1 and HER2), which can lead to a decrease in protein phosphorylation of both subunits in response to gefitinib.

Lapatinib treatment has been shown to induce apoptosis in trastuzumab-resistant breast cancer cells. Lapatinib induced apoptosis in SKBR3 therapy sensitive cell lines; however, the therapy resistant cells evade apoptosis in the presence of the treatment suggesting that not only are these cells resistant to the treatments, but prolonged therapy provides an environment optimal for avoiding apoptosis.

Even though gefitinib has been shown to induce apoptosis in other cancer cell types, including breast cancer, the SKBR3 cells did not respond to gefitinib treatment via apoptosis. This has also been confirmed by other studies where the apoptotic response to gefitinb was cell type-dependent. This lack of response may be because autophagy and not apoptosis has been shown to be an early response to gefitinib treatment in SKBR3 cells.

In addition to evasion of apoptosis, cancer cells undergo EMT during metastatic progression, which may produce subpopulations of cells with stem cell-like characteristics that contribute to therapy resistance. The SKBR3 therapy sensitive cells respond to gefitinib or lapatinib treatment with lower MFE used as a measure of stem cell-like activities, whereas TKI treatment had no effect in the therapy resistant cells. Moreover, an increase in MFE and established breast cancer stem cell markers in cells resistant to the higher concentration of gefitinib was observed, suggesting that the therapy resistant breast cancer cells may have more cancer stem cell activity that can contribute to therapy resistance.

Similar to trastuzumab, lapatinib resistance results in circumvention of the kinase inhibitory function by acquiring point mutations in HER2 and EGFR, as well as via elevated downstream signaling. Therefore, activation of compensatory pathways downstream of EGFR and HER2 is a common mechanism of resistance to lapatinib and gefitinib therapy. Central to these pathways are the activation of Akt via PI-3K and the Ras/MAPK pathway. However, when investigating potential mechanisms of therapy resistance and the possible activation of compensatory pathways it was shown that Akt and MAPK activities (Phosphorylation) were unchanged in the therapy resistant SKBR3 cells.

Expression and activity of the Rho GTPase Rac, but not related family members RhoA and Cdc42, are elevated in the therapy resistant variants. The Rho GTPase family is known to regulate therapy resistance and CSC maintenance. Of the Rho GTPases, Rac has been implicated with cancer therapy resistance, specifically via the oncogenic guanine nucleotide exchange factors that are coupled to EGFR and HER2 signaling. Numerous studies have implicated Rac/PAK activities with the maintenance of mesenchymal stem cell-like populations in epithelial cancers; and thus, therapy resistance, especially in HER2-type breast cancer. Accordingly, results presented herein with the Rac inhibitors EHop-016 and MBQ-167 show that both these inhibitors significantly reduce the MFE of HER2+ and EGFR+breast cancer cells. Moreover, The Cancer Genome Atlas (TCGA) data show that Rac1 or PAK1 overexpression is associated with malignant breast cancer and significantly diminishes HER2 type patient survival within 10 years following diagnosis. Similar to present finding that Rac1 is overexpressed in therapy resistant variants of breast cancer cells, Rac1 has also been shown to be overexpressed in naturally occurring lapatinib-resistant HER2 type breast cancer cell lines. Therefore, it is likely that that Rac1 inhibition is a rational strategy for sensitization of lapatinib and gefitinib resistant tumors.

Accordingly, in the therapy resistant variants discovered herein, the Rac inhibitor EHop-016, which was designed and developed by us to inhibit Rac activation by the oncogene Vav, which is activated by EGFR/HER2, or the dual Rac1/Cdc42 inhibitor MBQ-167 (Humphries-Bickley et al., 2017), reduced viability and induced apoptosis in single or combined treatments with lapatinib or trastuzumab. Although there was a trend in further reduction of cell viability when the Rac inhibitor was combined with gefitinib, lapatinib, or trastuzumab in the therapy resistant variants, this effect was not additive or synergistic. However, data disclosed herein clearly shows the utility of using Rac inhibitors as a valid strategy to reduce viability of highly aggressive breast cancer cells. In a mouse model of metastasis, the highly metastatic and therapy resistant MDA-MB-435 variant used for the present disclosure, reduced mammary fat pad tumor growth by ˜85% and metastasis by 100%.

In support of a role for Rac inhibition in chemosensitization, Rac1 knockdown has been shown to sensitize lapatinib resistance, and a small molecule inhibitor of Rac1, NSC23766, was shown to increase sensitivity to the anti-HER2 therapeutic trastuzumab (Zhao et al.), overcome gefitinib resistance in non-small cell lung carcinoma, and be effective in combination therapy with eroltinib, another tyrosine kinase inhibitor. Additionally, EHop-016 sensitizes HER2 overexpressing trastuzumab sensitive and resistant breast cancer cells to trastuzumab, and was recently shown to overcome therapy resistance by combined cancer therapy with Akt/mTOR inhibitors. Therefore, targeting Rac is considered a viable strategy to overcome anti-EGFR/HER2 therapy resistance in cancer (Zhao et al, 2011; Dokmanovic et al., 2009).

The salient observation that the therapy resistant variants overexpress and activate Rac1, an established driver of metastasis, is highly relevant towards novel therapeutic strategies to overcome therapy resistance. Most studies illustrating the utility of Rac inhibitors have used the Tiam1/Rac inhibitor NSC23766, which is active at 50-100 μM concentrations, which are too high to be pharmacologically useful. The inventors (U.S. Pat. Nos. 9,981,980 and 10,392,396) found Rac inhibitors that act through disparate mechanisms, the Vav2/Rac inhibitor EHop-016 and the nucleotide association inhibitor MBQ-167, at 100× lower effective concentrations than NSC23766. EHop-016 and MBQ-167 were tested in mouse models of HER2+breast cancer and have demonstrated their utility as metastasis inhibitors (Humphries-Bickley et al., 2017). Therefore, these combined results signify the importance of Rac and its close homology to Cdc42 as viable targets to treat EGFR/HER2 targeted therapy resistant cancer.

### MATERIALS AND METHODS

Cell Culture

Metastatic human breast cancer cells SKBR3 (American Type Culture Collection) and metastatic cancer cell line MDA-MB-435 (provided by Dr. Danny Welch) were maintained in complete culture medium: Dulbecco's modified Eagle's medium (Invitrogen) supplemented with 10% fetal bovine serum (Invitrogen) at 37° C. in 5% CO₂. Gefitinib (Gef.R) and lapatinib resistant (Lap.R) variants were created from these EGFR/HER2 (+) gefitinib and lapatinib sensitive SKBR3 cells by exposing the sensitive cells to a range of concentrations up to 0.5 μM for ˜6 months. The cells that survived at concentrations >0.1 μM were selected as resistant variants.

Cell Viability

The CellTiter 96 Non-Radioactive Assay (Promega) was used according to manufacturer's instructions. Briefly, cells were seeded in a 24 well plate and treated for 48 hours with vehicle, gefitinib, lapatinib, trastuzumab, and (or) EHop-016 or MBQ-167 at the indicated concentrations. After incubation, the MTT (3-(4,5-dymethyl thiazol-2-yl)-2,5-diphenyl tetrazolium bromide) reagent was added to the plate (40 μL/well). The plates were incubated for 4h at 37° C., followed by the addition of stop solution, and the plates were incubated to facilitate solubilization of formed formazan salts. The absorbance was measured at 570 nm using a microplate reader. Fold resistance for therapy resistant cell lines was quantified, as described in (Montalvo-Ortiz et al., 2012), by the ratio of the half maximal inhibitory concentration (IC₅₀) of the therapy resistant cell line by the IC₅₀ of the therapy sensitive cells.

Caspase Assay

Apoptosis was analyzed by the Caspase-Glo 3/7 activity assay (Promega) as described by the manufacturer. Briefly, cells were seeded in a 96 well plate and treated for 48h. Luminogenic caspase- 3/7 substrate containing a DEVD sequence was added and incubated for 1h. The luminescence was measured by a plate-reading luminometer.

Western Blotting

Therapy sensitive and resistant variants were lysed and Western blotted using routine procedures. Briefly, equal total protein amounts from cell lysates were run on SDS-PAGE gels and Western blotted using specific antibodies against EGFR, pEGFR, HER2, pHER2, Integrin (33, Nanog, CD133, AKT, pAKT, MAPK, pMAPK and Rac. Anti-β-actin was used for normalization. The integrated density of positive bands of total and phospho EGFR/HER2 were quantified using Image J software, as per routine laboratory protocols (Martinez-Montemayor et al.).

Mammosphere Assay

A mammosphere assay was performed to determine cancer stem cell-like activity, as described in (Humphries-Bickley et al., 2017). SKBR3 cells were seeded in ultra-low attachment plates (Corning) at a density of 500 cells/well in serum-free mammary epithelium basal medium (Lonza) supplemented with 1% penicillin/streptomycin (Lonza), B27 supplement minus vitamin A (50×, Gibco), 5 μg/mL insulin (Gibco), 1 μg/mL hydrocortisone (Sigma), 20 ng/mL EGF, and 20 ng/mL fibroblast growth factor (Sigma). Mammospheres were counted using an inverted microscope after 4 days of incubation in 37° C., 5% CO₂. Mammosphere forming efficiency (MFE) was calculated as the number of mammospheres divided by the number of cells seeded per well and is expressed as a percentage.

Rac Activation Assay

Rac activity was analyzed from SKBR3 sensitive and resistant cell lysates by pull-down assays. The P21-binding domain (PBD) of PAK 1 was used to isolate active GTP-bound Rac, as described previously (Baugher et al., 2005). Active and total Rac GTPases were separated in a 12% SDS-PAGE gel and identified by Western blotting using Rac specific antibodies (Cell Signaling Technology, Inc).

Statistical Analysis

Statistical comparisons between therapy sensitive and resistant cell lines for SKBR3 cells resistant to gefitinib or lapatinib were conducted by Student's T test using GraphPad Prism 6. Differentially expressed genes and proteins were selected at >1.5-fold expression, statistical significance of p<0.05.

### Abbreviations

CSC: cancer stem cell
EMT: epithelial to mesenchymal transition
EGFR: epidermal growth receptor
Gef.R: gefitinib resistant
Lap.R: lapatinib resistant
HER2: human epidermal growth factor receptor
IC₅₀: half maximal inhibitory concentration
MAPK: mitogen activated protein kinase
MFE: mammosphere forming efficiency
mTOR: mammalian target of rapamycin
MTT: 3-(4,5-dymethyl thiazol-2-yl)-2,5-diphenyl tetrazolium bromide
p: phospho
PAK: p21-activated kinase
PI3K: phosphoinositide 3-kinase
TCGA: The Cancer Genome Atlas
TKI: tyrosine kinase inhibitor

### PUBLICATIONS CITED

The publications cited herein are incorporated by reference to the extent that they relate materials and methods relevant to the claimed invention.

Baugher P J, Krishnamoorthy L, Price J E, Dharmawardhane S F. Rac1 and Rac3 isoform activation is involved in the invasive and metastatic phenotype of human breast cancer cells. Breast Cancer Res. 2005;7:R965-74. doi:10.1186/bcr1329.

de Melo Gagliato D, Jardim D L F, Marchesi M S P, Hortobagyi G N. Mechanisms of resistance and sensitivity to anti-HER2 therapies in HER2+breast cancer. Oncotarget. 2016; 7:64431-46. doi:10.18632/oncotarget.7043.

Dokmanovic M, Hirsch D S, Shen Y, Wu W J. Rac1 contributes to trastuzumab resistance of breast cancer cells: Rac1 as a potential therapeutic target for the treatment of trastuzumab-resistant breast cancer. Mol.Cancer Ther. 2009, 8:1557-69. doi: 10.1158/1535-7163.MCT-09-0140.

Humphries-Bickley T, Castillo-Pichardo L, Hernandez-O-Farrill E, Borrero-Garcia L D, Forestier-Roman I, Gerena Y, et al. Characterization of a Dual Rac/Cdc42 Inhibitor MBQ-167 in Metastatic Cancer. Mol Cancer Ther. 2017;molcanther.0442.2016. doi:10.1158/1535-7163.MCT-16-0442.

Liu Q, Yu S, Zhao W, Qin S, Chu Q, Wu K. EGFR-TKIs resistance via EGFR-independent signaling pathways. Molecular Cancer. 2018; 17:53. doi:10.1186/s12943-018-0793-1.

Martinez-Montemayor M M, Otero-Franqui E, Martinez J, De L M-P, Cubano L A, Dharmawardhane S. Individual and combined soy isoflavones exert differential effects on metastatic cancer progression. Clin.Exp.Metastasis. 27:465-80. doi:10.1007/s10585-010-9336-x.

McDermott M, Eustace A J, Busschots S, Breen L, Crown J, Clynes M, et al. In vitro Development of Chemotherapy and Targeted Therapy Drug-Resistant Cancer Cell Lines: A Practical Guide with Case Studies. Front Oncol. 2014;4 March:40. doi:10.3389/fonc.2014.00040.

Montalvo-Ortiz B L, Castillo-Pichardo L, Hernández E, Humphries-Bickley T, De La Mota-Peynado A, Cubano L A, et al. Characterization of EHop-016, Novel Small Molecule Inhibitor of Rac GTPase. J Biol Chem. 2012; 287:13228-38. doi:10.1074/jbc.M111.334524.

Zhao Y, Wang Z, Jiang Y, Yang C. Inactivation of Rac1 reduces Trastuzumab resistance in PTEN deficient and insulin-like growth factor I receptor overexpressing human breast cancer SKBR3 cells. Cancer Lett. 2011; 313:54-63. doi:10.1016/j.canlet.2011.08.023.

We claim:

1. A method to overcome resistance to treatments targeted to Epidermal Growth Factor Receptor family member expression in breast cancer cells, the method comprising:
(a) selecting a treatment compound targeted to the Epidermal Growth Factor Receptor family member;
(b) selecting a Rac inhibitor;
(c) contacting the breast cancer cells with either a combination of the selected treatment compound and the selected Rac inhibitor, or with the treatment compound and the Rac inhibitor administered sequentially.

2. The method of claim 1, wherein the treatments are selected from a group consisting of Gefitinib®, Lapatinib®, and combinations thereof.

3. The method of claim 1, wherein the Epidermal Growth Factor Receptor is EGFR/HER2.

4. The method of claim 1, wherein the Rac inhibitor is a 1,5-disubstituted 1, 2, 3-triazoles.

5. The method of claim 4, wherein the Rac inhibitor is selected from the group consisting of EHop-016, MBQ-167 and combinations thereof.

6. A combination of compounds comprising a Rac and inhibitor with therapeutic compounds targeted at EGFR/HER2 receptors.

7. The combination of claim 6, wherein the compounds inhibiting Rac are selected from the group consisting of EHop-016, MBQ-167 and combinations thereof.

8. A method to treat EGFR/HER2 resistant breast cancer in a patient in need thereof, the method comprising:
(a) obtaining EHop-016 or MBQ-167;
(b) combining EHop-016 or MBQ-167 with gefitinib or lapatinib or the equivalent; and
(c) administering the combination to the patient.
